(19) United States
(12) Patent Application Publication
(10) Pub. No.: US 2010/0331659 A1
(43) Pub. Date: Dec. 30, 2010

(54) LOW ARTIFACT DEFIBRILLATION ELECTRODE

(76) Inventors: DAVID A. SHERATON, SR., San Clemente, CA (US); James Perrault, Carlsbad, CA (US)

Correspondence Address: CHARLES C.H. WU, 98 DISCOVERY, IRVINE, CA, 92618-3105, US

(21) Appl. No.: 12/494,027

(22) Filed: Jun. 29, 2009

(51) Int. Cl. A61B 5/04; A61N 1/04

(52) U.S. Cl. 600/382; 600/392; 600/395; 607/4

(57) ABSTRACT

An electrode arrangement for low artifact electrocardiogram (ECG) monitoring and defibrillation, comprises at least one conductor print line body having a large silver conductor print line area functioning as a defibrillation area and a small silver-silver chloride (Ag/AgCl) conductor print line area functioning as a sensing area, at least one non halide defibrillation gel layer and halide containing sensing gel layer configured to attach with the defibrillation area and sensing area respectively, and at least one insulated conductive ink line for connecting the conductor print line body to a cable that provides an electrical communication between the conductor print line body and an external equipment. When direct current runs through the conductor print line body, the large silver conductor print line area polarizes and the small Ag/AgCl conductor print line area does not polarize so that small Ag/AgCl conductor print line area senses ECG signal leading to low motion artifact.

### BACKGROUND OF THE INVENTION

1. Technical Field of the Invention

The present invention relates in general to an electrode arrangement for defibrillation and electrocardiogram (ECG) monitoring. More specifically, the present invention relates to an electrode arrangement having separate areas for defibrillation and low artifact ECG monitoring.

2. Description of the Related Art

Defibrillator pads are generally used for the management of life-threatening cardiac arrhythmias causing by abnormal electrical activity in the heart, characterized by irregular contractions. Today, with the advance of technology, portable defibrillators are no longer bulky devices and can be placed in ambulances, automated units placed in a public area, or even wearable units placed directly unto a patient's body. Of relatively recent improvements are electrode pads which comprise a multiplicity of electrodes affixed to a flexible pad in an appropriate pattern for use in medical analysis and/or therapy.

Traditional single element electrode pads are made of one thin flexible conductor sandwiched between a protective polymer backing and a hydro-gel adhesive that can be used for either monitoring a patient's heart condition or stimulating electrical impulses from a cardiac system to the patient's body. A common problem associated with the use of such single element electrode pads is, whether for stimulating or monitoring, the pads are constructed similarly which may cause false recognition of electrocardiogram (ECG) signals.

Another known type of external defibrillator has a pair of disposable electrodes configured to be adhered to the skin of a patient, each electrode including an electrically conductive layer comprising a metal that is polarized during a defibrillating pulse, and a control unit configured to deliver a defibrillating pulse to the patient through the electrodes and to generate the ECG from the electrical signals. A waveform is configured to substantially depolarize the metal, and may be, for example, a biphasic waveform. The problem with such defibrillator is that, the depolarization is corrected by the current flow. Moreover, if the metal does not depolarize quickly after delivering the defibrillating pulse to the patient, the electrode will not be capable of sensing the signal to allow the defibrillator to generate a clear ECG and determine whether another shock should be delivered within a short period of time.

Some other bioelectric interface provides a plurality of electrodes affixed to an adhesive sheet which demonstrates electrode isolating anisotropic electrical specific impedance properties simultaneous with isotropic pliability and adhesion mechanical properties. The bioelectric interface is a twelve lead (ECG) system. Multiple leads are unwieldy and cumbersome since the leads from the bioelectric interface often become twisted. The twisting of the leads is problematic since it may result in delays in delivering necessary emergency care to the patient.

In order to address the need of sequential defibrillating and monitoring from a single set of electrodes, many attempts have been proposed in the past. One such proposed system includes physically separated stimulating and monitoring gel pads, but did not address any solution to solve the problem of motion artifact.

Hence, it can be seen, that there is a need for a single electrode arrangement having separate areas for defibrillation and low artifact ECG monitoring. Further, the needed arrangement would be simple, economical, and would deliver electrical impulse to a patient's heart and sequentially would be able to convey an ECG signal from the patient's heart to the external monitoring equipment. Moreover, the needed arrangement would utilize minimum number of leads that are easy to manipulate to avoid twisting of the leads when in use.

### SUMMARY OF THE INVENTION

To minimize the limitations found in the prior art, and to minimize other limitations that will be apparent upon the reading of the specifications, the present invention provides an electrode arrangement for low artifact electrocardiogram (ECG) monitoring and defibrillation. The electrode arrangement comprises at least one conductor print line body having a large silver conductor print line area functioning as a defibrillation area and a small silver-silver chloride (Ag/AgCl) conductor print line area functioning as a sensing area, at least one non-chloride defibrillation gel layer configured to attach with the defibrillation area on a bottom side of the conductor print line body, at least one chloride containing sensing gel layer configured to attach with the sensing area on the bottom side of the conductor print line body, whereby as a direct current (DC) runs through the conductor print line body, the large silver conductor print line area polarizes and the small Ag/AgCl conductor print line area does not polarize so that the small Ag/AgCl conductor print line area is picked up first and senses an ECG signal.

In another aspect of the present invention, a method in accordance with the present invention is a method of treating a patient with an electrode arrangement for low artifact electrocardiogram (ECG) monitoring and defibrillation, comprising of providing the electrode arrangement comprising a conductor print line body having a defibrillation area and a sensing area, removing a cover for a skin contacting side and applying the electrode arrangement that is configured to adhesively secure to the skin of the patient, receiving a first set of electrical impulses from an external stimulating equipment on the defibrillation area and delivering the first set of electrical impulses to a patient's heart, and sequentially receiving a second set of electrical impulses on the sensing area from the patient's heart and conveying the second set of electrical impulses to an external monitoring equipment.

One objective of the invention is to provide an electrode arrangement producing significantly less motion artifact in an electrocardiogram (ECG) signal due to a small conductive surface area of the ECG sensing area.

Another objective of the invention is to provide an electrode arrangement having a defibrillating area and an electrocardiogram (ECG) sensing area, wherein the defibrillation area being able to function as any of a plurality of other cardiac stimulation area including pacing, cardioversion, etc.

A third objective of the invention is to provide an electrode arrangement having a large defibrillation area and a smaller sensing area that can be connected by one single wire for electrical communication with external equipment.

Yet another objective of the invention is to provide a simple electrode arrangement capable of two lead electrocardiogram (ECG) monitoring.

It is also the objective of the invention to provide an electrode arrangement for low artifact ECG monitoring and defibrillation that minimizes or eliminates the problem of poor adhesion, burns, and movement of electrodes.

These and other advantages and features of the present invention are described with specificity so as to make the present invention understandable to one of ordinary skill in the art.

### BRIEF DESCRIPTION OF THE DRAWINGS

Elements in the figures have not necessarily been drawn to scale in order to enhance their clarity and improve understanding of these various elements and embodiments of the invention. Furthermore, elements that are known to be common and well understood to those in the industry are not depicted in order to provide a clear view of the various embodiments of the invention, thus the drawings are generalized in form in the interest of clarity and conciseness.

FIG. 1 is an exploded perspective view of the preferred embodiment in accordance with the present invention;

FIG. 2 is a schematic layout of the preferred embodiment in accordance with the present invention showing the large defibrillation area and the central sensing area being connected by one wire;

FIG. 3 is an operational flow chart of the preferred embodiment in accordance with the present invention illustrating the method of treating the patient with the electrode arrangement;

FIG. 4 is an exploded perspective view of another embodiment; and

FIG. 5 is a schematic layout of another embodiment showing the large defibrillation area and the central sensing area being electrically isolated from each other.

### DETAILED DESCRIPTION OF THE DRAWINGS

In the following discussion that addresses a number of embodiments and applications of the present invention, reference is made to the accompanying drawings that form a part hereof, and in which is shown by way of illustration specific embodiments in which the invention may be practiced. It is to be understood that other embodiments may be utilized and changes may be made without departing from the scope of the present invention.

Various inventive features are described below that can each be used independently of one another or in combination with other features. However, any single inventive feature may not address any of the problems discussed above or only address one of the problems discussed above. Further, one or more of the problems discussed above may not be fully addressed by any of the features described below.

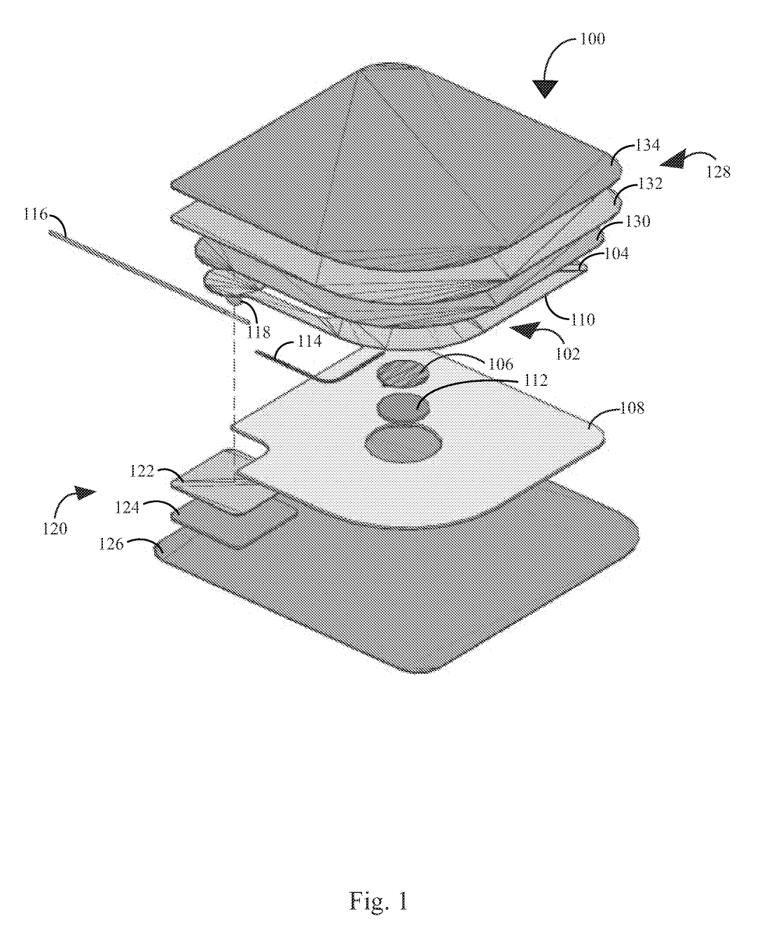

FIG. 1 is an exploded perspective view of the preferred embodiment in accordance with the present invention showing an electrode arrangement 100 for low artifact electrocardiogram (ECG) monitoring and defibrillation. The electrode arrangement 100 comprises of at least one conductor print line body 102 having a large silver conductor print line area functioning as a defibrillation area 104 and a small silver-silver chloride (Ag/AgCl) conductor print line area functioning as a central sensing area 106. The central sensing area 106 is located centrally of the defibrillation area 104. The electrode arrangement 100 further comprises of at least one non-chloride defibrillation gel layer 108 configured to attach with the defibrillation area 104 on a bottom side of the conductor print line body 110, at least one chloride containing sensing gel layer 112 configured to attach with the central sensing area 106 on the bottom side of the conductor print line body 110, at least one black carbon ink 114 for connecting the conductor print line body 102 to a cable 116 that provides an electrical communication between the conductor print line body 102 and an external equipment (not shown). At least one conductive adhesive 118 is further included that connects the cable 116 to the conductor print line body 102. When a direct current (DC) runs through the conductor print line body 102, the large silver conductor print line area 104 polarizes and the small Ag/AgCl conductor print line area 106 does not polarize, so that the small Ag/AgCl conductor print line area 106 is picked up first and senses an ECG signal.

A motion artifact in the ECG signal may be significantly less due to a small conductive surface area of the central sensing area 106. It is important for the health of a patient that an external stimulating equipment (not shown), for example an automatic external defibrillator (AED) properly diagnosis a shockable arrhythmia from a non-shockable ECG or healthy ECG. The motion artifact from cardiopulmonary resuscitation (CPR), lead wire jerking, etc can cause false recognition of ventricular tachycardia and ventricular fibrillation followed by unnecessary, harmful, and potentially lethal shocks.

The conductor print line body 102 has a plurality of top layers 128 that include a substrate for conductor print line body 130, a laminating adhesive 132, and an insulated backing member 134. Further, the conductor print line body 102 has a plurality of bottom layers 120 that include a laminating adhesive 122, a connection insulation 124, and a removable cover for skin contacting side 126.

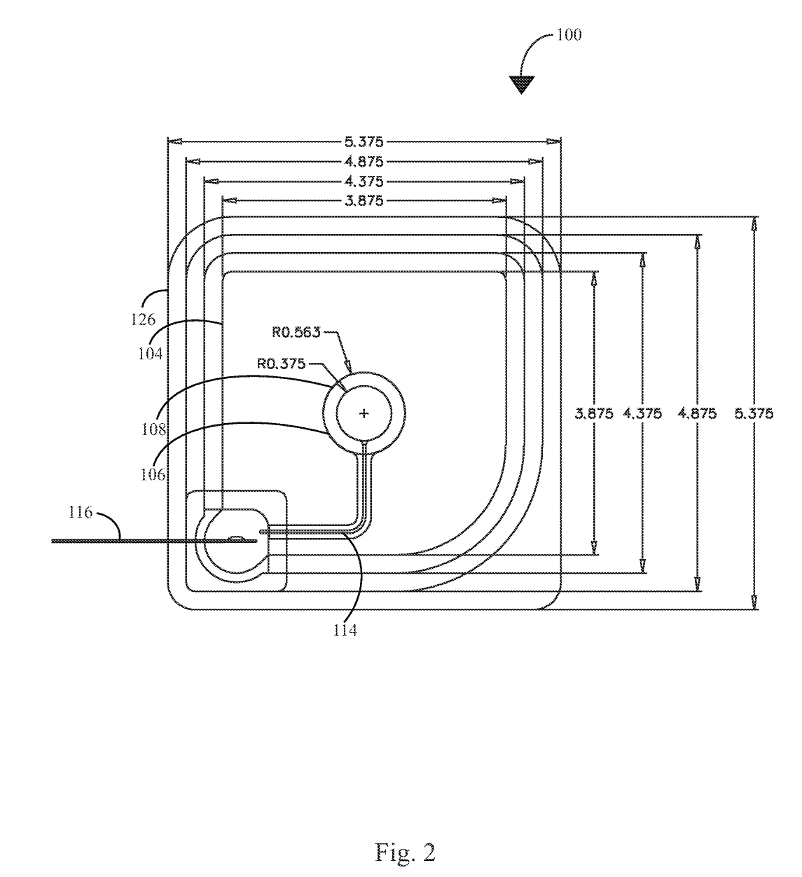

FIG. 2 shows a schematic layout of the electrode arrangement 100. In this current exemplary embodiment, the large defibrillation area 104 and the central sensing area 106 can be connected by one single wire for electrical communication with the external equipment (not shown), since the small central sensing area 106 is Ag/AgCl and the defibrillation area 104 is only silver. Moreover, by printing separate silver areas on a common substrate 130, it is more economical than individual ECG monitoring and defibrillation electrodes. The defibrillation area 104 delivers an electrical impulse to a patient's heart and the central sensing area 106 sequentially conveys the ECG signal from the patient's heart to external monitoring equipment. The defibrillation area 104 can function as any of a plurality of other cardiac stimulation area including pacing, cardioversion, etc. The electrode arrangement 100 of the preferred embodiment is capable of two lead ECG monitoring.

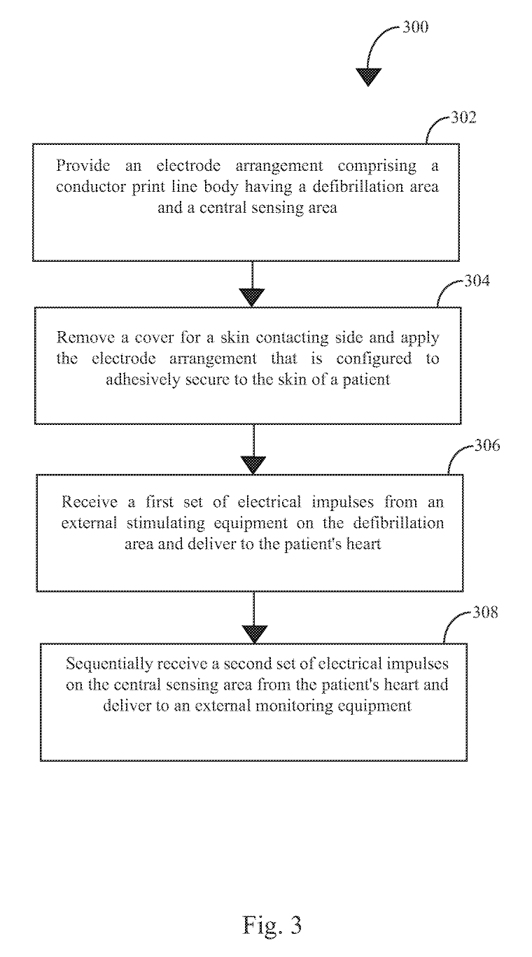

FIG. 3 shows an operational flow chart of the preferred embodiment in accordance with the present invention illustrating a method of treating the patient with the electrode arrangement 300 for low artifact ECG monitoring and defibrillation. The electrode arrangement comprising the conductor print line body having the defibrillation area and the central sensing area is provided as indicated at block 302. The cover for a skin contacting side is removed and the electrode arrangement that is configured to adhesively secure to the skin of the patient is applied as indicated at block 304. A first set of electrical impulses from the external stimulating equipment is received on the defibrillation area and delivered to the patient's heart as indicated at block 306. A second set of electrical impulses is received sequentially on the central sensing area from the patient's heart and delivered to the external monitoring equipment as indicated at block 308.

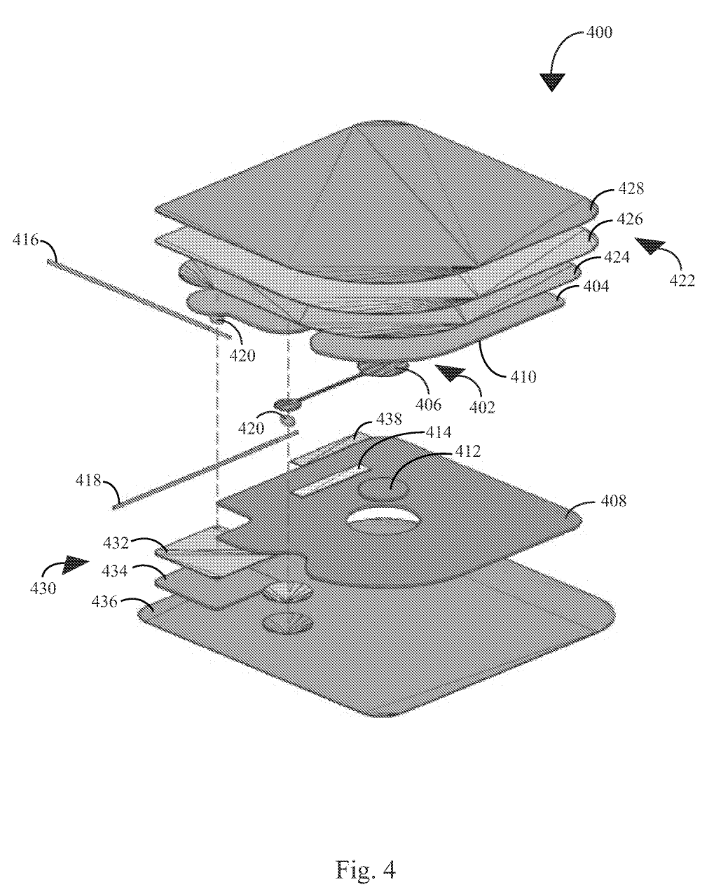

In another embodiment of the invention, there may have an electrically isolated defibrillation area and central sensing area that can be wired separately for the electrical communication with the external stimulating equipment and the external monitoring equipment respectively. For example, FIG. 4 is an exploded perspective view of another embodiment of an electrode arrangement 400 for low artifact ECG monitoring and defibrillation. The electrode arrangement 400 comprises of at least one Ag/AgCl conductor print line body 402 having a large Ag/AgCl print line area functioning as a defibrillation area 404 and a small Ag/AgCl print line area functioning as a central sensing area 406. The central sensing area 406 is located centrally of the defibrillation area 404 and both are electrically isolated from each other. The electrode arrangement 400 further comprises of at least one defibrillation gel layer 408 containing chloride configured to attach with the defibrillation area 404 on a bottom side of the Ag/AgCl conductor print line body 410, at least one sensing gel layer 412 containing chloride configured to attach with the central sensing area 406 on the bottom side of the Ag/AgCl conductor print line body 410, at least one insulator sheet 414 located over the defibrillation gel layer 408 for insulating the central sensing area 406 from the defibrillation gel layer 408, at least one defibrillation wire 416 configured to provide the electrical communication between the defibrillation area 404 and the external stimulating equipment (not shown), and at least one sensing wire 418 configured to provide the electrical communication between the central sensing area 406 and the external monitoring equipment (not shown). The at least one insulator sheet 414 can be a plastic film, printable resin, or ceramic.

As in FIG. 4, at least one pair of conductive adhesive 420 is further included that connects the defibrillation area 404 and the central sensing area 406 to the defibrillation wire 416 and the sensing wire 418 respectively. A conductive surface area of the central sensing area 406 is small in size, hence the motion artifact is significantly less for ECG monitoring. The Ag/AgCl conductor print line body 402 includes a plurality of top layers 422 including a substrate for conductor print 424, a laminating adhesive 426, and an insulated backing member 428. The Ag/AgCl conductor print line body 402 further includes a plurality of bottom layers 430 including a laminating adhesive 432, a connection insulation 434, and a removable cover for skin contacting side 436.

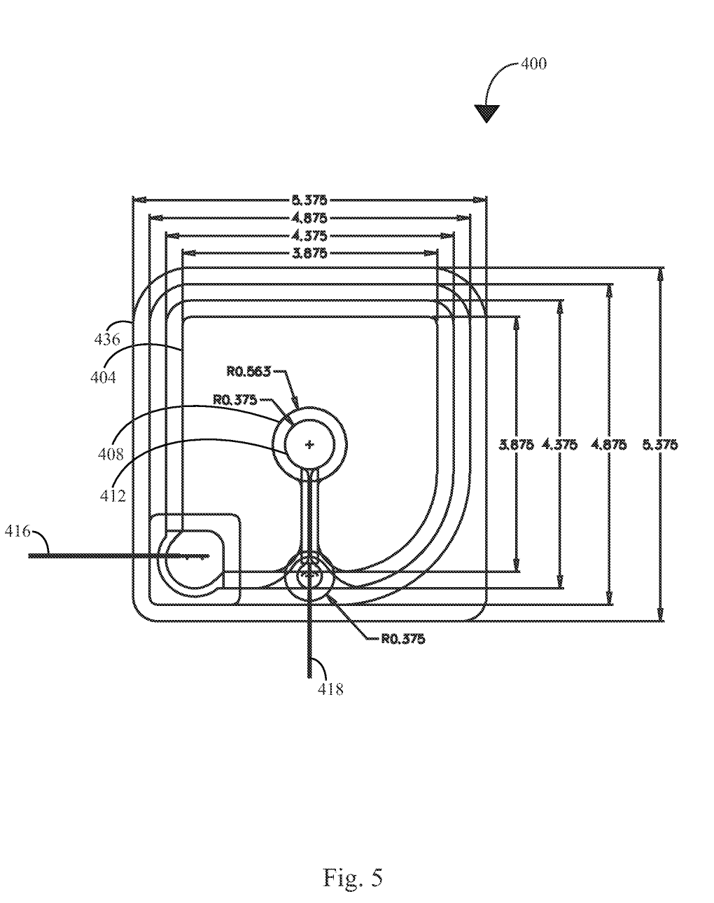

The connections are separate for defibrillation and ECG monitoring. Both the defibrillation gel layer 408 and the sensing gel layer 412 can have same chloride containing formula as the two Ag/AgCl print line areas (404, 406) are physically separated. The at least one insulator sheet 414 located over the defibrillation gel layer 408 is covered with an insulating laminating adhesive 438 for insulating the central sensing area 406 from the defibrillation gel layer 408. FIG. 5 is a schematic layout of another embodiment of an electrode arrangement 400 shown in FIG. 4.

The foregoing description of the preferred embodiment of the present invention has been presented for the purpose of illustration and description. It is not intended to be exhaustive or to limit the invention to the precise form disclosed. Many modifications and variations are possible in light of the above teachings. It is intended that the scope of the present invention not be limited by this detailed description, but by the claims and the equivalents to the claims appended hereto.

What is claimed is:

1. An electrode arrangement for low artifact electrocardiogram (ECG) monitoring and defibrillation, comprising:
at least one conductor print line body having a large silver conductor print line area functioning as a defibrillation area and a small silver-silver chloride (Ag/AgCl) conductor print line area functioning as a sensing area;
at least one non halide defibrillation gel layer configured to attach with the defibrillation area on a bottom side of the conductor print line body;
at least one halide containing sensing gel layer configured to attach with the sensing area on the bottom side of the conductor print line body;
at least one insulated conductive ink line for connecting the conductor print line body to a cable that provides an electrical communication between the conductor print line body and an external equipment; and
whereby as a direct current (DC) runs through the conductor print line body, the large silver conductor print line area polarizes and the small Ag/AgCl conductor print line area does not polarize so that the small Ag/AgCl conductor print line area is picked up first and senses an ECG signal.

2. The electrode arrangement of claim 1 wherein a motion artifact in the ECG signal may be significantly less due to a small conductive surface area of the sensing area.

3. The electrode arrangement of claim 1 wherein the defibrillation area delivers an electrical impulse to a patient's heart and the sensing area sequentially conveys the ECG signal from the patient's heart to external monitoring equipment.

4. The electrode arrangement of claim 1 wherein the conductor print line body comprises a plurality of top layers including a substrate for conductor print line body, a laminating adhesive, and an insulated backing member.

5. The electrode arrangement of claim 1 wherein the conductor print line body further comprises a plurality of bottom layers including a laminating adhesive, a connection insulation, and a removable cover for skin contacting side.

6. The electrode arrangement of claim 1 wherein the defibrillation area can function as any of a plurality of other cardiac stimulation area including pacing, cardioversion, etc.

7. A method of treating a patient with an electrode arrangement for low artifact electrocardiogram (ECG) monitoring and defibrillation, comprising the steps of:
providing the electrode arrangement comprising a conductor print line body having a defibrillation area and a sensing area;
removing a cover for a skin contacting side and applying the electrode arrangement that is configured to adhesively secure to the skin of the patient;
receiving a first set of electrical impulses from an external stimulating equipment on the defibrillation area and delivering the first set of electrical impulses to a patient's heart; and
sequentially receiving a second set of electrical impulses on the sensing area from the patient's heart and conveying the second set of electrical impulses to an external monitoring equipment.

8. The method of claim 7 wherein the conductor print line body comprises a plurality of top layers including a substrate for conductor print line body, a laminating adhesive, and an insulated backing member.

9. The method of claim 7 wherein the conductor print line body comprises a plurality of bottom layers including a laminating adhesive, a connection insulation, and a removable cover for skin contacting side.

10. The method of claim 7 wherein the defibrillation area can function as any of a plurality of other cardiac stimulation area including pacing, cardioversion, etc.
